(19) United States
(12) Patent Application Publication
(10) Pub. No.: US 2009/0104238 A1
(43) Pub. Date: Apr. 23, 2009

(54) METHOD FOR PRODUCING POLYMERS MODIFIED BY SILICONE

(75) Inventors: Kurt Stark, Neuhaus (DE); Christian Hogl, Reut (DE)

Correspondence Address: BROOKS KUSHMAN P.C., 1000 TOWN CENTER, TWENTY-SECOND FLOOR, SOUTHFIELD, MI, 48075, US

(73) Assignee: WACKER CHEMIE AG, Munich (DE)

(21) Appl. No.: 11/718,647

(22) PCT Filed: Oct. 20, 2005
(86) PCT No.: PCT/EP2005/011301

§ 371 Date: May 10, 2007

(30) Foreign Application Priority Data
Nov. 4, 2004 (DE) 10 2004 053 314.8

(51) Int. Cl. A61K 8/04; C08L 83/04; C08F 283/12; G03G 9/087

(52) U.S. Cl. 424/401; 524/547; 526/279; 430/109.3

(57) ABSTRACT

Silicone-modified polymers of ethylenically unsaturated monomers are prepared in the form of aqueous polymer dispersions or water-redispersible polymer powders, by
A) preparing a prepolymer by means of polymerization of one or more ethylenically unsaturated monomers and at least one ethylenically unsaturated silicone macromer and isolating the prepolymer,
B) dissolving the prepolymer in one or more ethylenically unsaturated monomers,
C) emulsifying this solution in water and polymerizing by a free-radical mechanism, and optionally
D) drying the resulting aqueous dispersion of silicone-modified polymers.

The invention relates to a process for preparing silicone-modified polymers of ethylenically unsaturated monomers in the form of their aqueous polymer dispersions or water-redispersible polymer powders.

Various processes for preparing polymer dispersions of silicone-modified copolymers are known from the prior art. EP-A 771826 discloses a process for preparing a crosslinked silicone copolymer latex, in which water, monomer, emulsifier and water-soluble initiator are firstly placed in a reaction vessel, the reaction is started, further monomer is slowly metered in and the crosslinking silicone, which has short chains and is multiply unsaturated, is finally added together with the residual monomer.

In EP-A 614924, only short-chain silicone macromers are used in the emulsion polymerization, since relatively long-chain silicone macromers do not polymerize sensibly with the organic monomers. The silicone which contains free-radically polymerizable groups and the vinyl monomer are each emulsified in the aqueous phase and the polymerization is started. U.S. Pat. No. 6,602,949 describes the preparation of silicone-organopolymer graft polymers, in which a branched, short-chain silicone having a dendritic structure and having an ethylenically unsaturated radical, ethylenically unsaturated monomer and a free-radically polymerizable emulsifier are reacted in the presence of an oil-soluble initiator. The dendritic structure of the short-chain silicone macromer improves the copolymerization with organic monomers. In comparative examples, it is shown that long-chain silicone macromers (without a dendritic structure) have polymerized with organic monomers to an extent of only up to a maximum of 75% and a large amount of unreacted silicone macromer is left over. In EP-A 810243, silicone macromers are polymerized with organic monomers in emulsion, with use being made exclusively of an oil-soluble initiator. A disadvantage of processes initiated by an oil-soluble initiator is the unsatisfactory stability of the resulting dispersions, which display a very strong tendency to undergo phase separation.

U.S. Pat. No. 5,618,879 describes the copolymerization of a mixture of silicone macromer and monomer emulsified in water by means of an anionic emulsifier, with the polymerization being initiated by a water-soluble initiator. In JP-A 05-140255, a silicone macromer containing free-radically polymerizable groups is dissolved in the organomonomer, the solution is emulsified in water by means of an anionic emulsifier and the polymerization is started by a water-soluble initiator. Here too, a disadvantage is that a considerable proportion of over 20% of the silicone macromer is not copolymerized.

JP-A 09-052923 describes a process for preparing silicone-containing graft polymers, in which a mixture of organopolysiloxane and ethylenically unsaturated silane is polymerized and vinyl monomer is subsequently added in two stages for grafting.

In all the processes known from the prior art, the copolymerization of the silicone macromers with organic monomers in emulsion always proceeds only to an insufficient extent. This leads to free silicone remaining in the dispersion, with the following associated disadvantageous consequences. The silicone migrates from coatings or films. The dispersion can coagulate. The particle size distribution is inhomogeneous. In addition, the tendency to undergo phase separation has an adverse effect on the storage stability.

It was therefore an object of the invention to provide silicone-modified polymers of ethylenically unsaturated monomers in which the silicone component is present in a form which very largely prevents migration of free silicone.

The invention provides a process for preparing silicone-modified polymers of ethylenically unsaturated monomers in the form of their aqueous polymer dispersions or water-redispersible polymer powders, characterized in that

A) a prepolymer is prepared by means of polymerization of one or more ethylenically unsaturated monomers and at least one silicone macromer having ethylenically unsaturated groups and is isolated,
B) the resulting prepolymer is dissolved in one or more ethylenically unsaturated monomers,
C) this solution is emulsified in water and polymerized by a free-radical mechanism, and, if appropriate,
D) the resulting aqueous dispersion of silicone-modified polymers is dried.

As ethylenically unsaturated monomers for the preparation of the prepolymer, use is made of one or more monomers from the group consisting of vinyl esters of unbranched or branched alkylcarboxylic acids having from 1 to 15 carbon atoms, methacrylic esters and acrylic esters of alcohols having from 1 to 15 carbon atoms, vinylaromatics, olefins, dienes and vinyl halides. In general, from 1 to 99% by weight, preferably from 40 to 95% by weight, of the ethylenically unsaturated monomers is used, in each case based on the total weight of the silicone macromer and monomer.

Suitable vinyl esters are vinyl esters of unbranched or branched carboxylic acids having from 1 to 15 carbon atoms. Preferred vinyl esters are vinyl acetate, vinyl propionate, vinyl butyrate, vinyl-2-ethylhexanoate, vinyl laurate, 1-methylvinyl acetate, vinyl pivalate and vinyl esters of α-branched monocarboxylic acids having from 5 to 13 carbon atoms, for example VeoVa9^{R} or VeoVa10^{R} (trade names of Resolution Performance Products). Particular preference is given to vinyl acetate.

Suitable monomers from the group consisting of esters of acrylic acid or methacrylic acid are esters of unbranched or branched alcohols having from 1 to 15 carbon atoms. Preferred methacrylic esters or acrylic esters are methyl acrylate, methyl methacrylate, ethyl acrylate, ethyl methacrylate, propyl acrylate, propyl methacrylate, n-butyl acrylate, isobutyl acrylate and t-butyl acrylate, n-butyl methacrylate, isobutyl methacrylate and t-butyl methacrylate, 2-ethylhexyl acrylate, norbornyl acrylate. Particular preference is given to methyl acrylate, methyl methacrylate, n-butyl acrylate, isobutyl acrylate and t-butyl acrylate, 2-ethylhexyl acrylate and norbornyl acrylate.

Suitable dienes are 1,3-butadiene and isoprene. Examples of copolymerizable olefins are ethene and propene. As vinylaromatics, it is possible to copolymerize styrene and vinyltoluene. As representatives of the group consisting of vinyl halides, vinyl chloride, vinylidene chloride or vinyl fluoride, preferably vinyl chloride, are usually used.

If appropriate, from 0.05 to 30% by weight, based on the total weight of the ethylenically unsaturated monomers, of auxiliary monomers can additionally be copolymerized. Examples of auxiliary monomers are ethylenically unsaturated monocarboxylic and dicarboxylic acids or salts thereof, preferably crotonic acid, acrylic acid, methacrylic acid, fumaric acid and maleic acid; ethylenically unsaturated carboxamides and nitriles, preferably acrylamide and acrylonitrile; monoesters and diesters of fumaric acid and maleic acid, e.g. the diethyl and diisopropyl esters, and also maleic anhydride, ethylenically unsaturated sulphonic acids or salts thereof, preferably vinylsulphonic acid, 2-acrylamido-2-methylpropanesulphonic acid. Further suitable auxiliary monomers are cationic monomers such as diallyldimethylammonium chloride (DADMAC), 3-trimethylammoniopropyl(meth)acrylamide chloride (MAPTAC) and 2-trimethylammonioethyl (meth)acrylate chloride. Vinyl ethers, vinyl ketones, further vinylaromatic compounds which may also have heteroatoms are also suitable as auxiliary monomers.

Further suitable auxiliary monomers are polymerizable silanes or mercaptosilanes. Preference is given to gamma-acryloxy-propyltri(alkoxy)silanes or gamma-methacryloxypropyltri-(alkoxy)silanes, α-methacryloxymethyltri(alkoxy)silanes, gamma-methacryloxypropylmethyldi(alkoxy)silanes, vinylalkyl-di(alkoxy)silanes and vinyltri(alkoxy)silanes, with alkoxy groups which can be used being, for example, methoxy, ethoxy, methoxyethylene, ethoxyethylene, methoxypropylene glycol ether or ethoxypropylene glycol ether radicals. Examples are vinyltrimethoxysilane, vinyltriethoxysilane, vinyltripropoxysilane, vinyltriisopropoxysilane, vinyltris(1-methoxy)iso-propoxysilane, vinyltributoxysilane, vinyltriacetoxysilane, 3-methacryloxypropyltrimethoxysilane, 3-methacryloxypropyl-methyldimethoxysilane, methacryloxymethyltrimethoxysilane, 3-methacryloxypropyltris(2-methoxyethoxy)silane, vinyltrichlorosilane, vinylmethyldichlorosilane, vinyltris-(2-methoxyethoxy)silane, trisacetoxyvinylsilane, 3-(triethoxysilyl)propyl(succinic anhydride)silane. Preference is also given to 3-mercaptopropyltriethoxysilane, 3-mercaptopropyl-trimethoxysilane and 3-mercaptopropylmethyldimethoxysilane.

Further examples are functionalized (meth)acrylates and functionalized allyl and vinyl ethers, in particular epoxy-functional compounds such as glycidyl acrylate, glycidyl methacrylate, allyl glycidyl ether, vinyl glycidyl ether, or hydroxyalkyl-functional compounds such as hydroxyethyl (meth)acrylate, or substituted or unsubstituted aminoalkyl (meth)acrylates, or cyclic monomers such as N-vinyl-pyrrolidone; or N-vinylformamide.

Further examples of suitable auxiliary monomers are precross-linking comonomers such as multiply ethylenically unsaturated comonomers, for example divinyl adipate, divinylbenzene, diallyl maleate, allyl methacrylate, butanediol diacrylate or triallyl cyanurate, or post-crosslinking comonomers, for example acrylamidoglycolic acid (AGA), methyl methylacryl-amidoglycolate (MMAG), N-methylolacrylamide (NMA), N-methylol-methacrylamide, N-methylolallyl carbamate, alkyl ethers such as isobutoxy ether or esters of N-methylolacrylamide, of N-methylolmethacrylamide and of N-methylolallyl carbamate.

Silicone macromers suitable for the preparation of the prepolymer are linear, branched, cyclic and three-dimensionally crosslinked polysiloxanes having at least 10 siloxane repeating units and at least one free-radically polymerizable functional group. The chain length is preferably from 10 to 10 000 siloxane repeating units. Ethylenically unsaturated groups such as alkenyl groups are preferred as polymerizable, functional groups.

Preferred silicone macromers are silicones having the general formula R¹ₐR₃₋ₐSiO(SiR₂O)ₙSiR₃₋ₐR¹ₐ, where the radicals R are identical or different and are each a monovalent, substituted or unsubstituted alkyl radical or alkoxy radical having from 1 to 18 carbon atoms in each case, R¹ is a polymerizable group, a is 0 or 1 and n=10 to 10 000.

In the general formula R¹ₐR₃₋ₐSiO(SiR₂O)ₙSiR₃₋ₐR¹ₐ, examples of radicals R are the methyl, ethyl, n-propyl, isopropyl, 1-n-butyl, 2-n-butyl, isobutyl, tert-butyl, n-pentyl, isopentyl, neopentyl, tert-pentyl radical, hexyl radicals such as the n-hexyl radical, heptyl radicals such as the n-heptyl radical, octyl radicals such as the n-octyl radical and isooctyl radicals such as the 2,2,4-trimethylpentyl radical, nonyl radicals such as the n-nonyl radical, decyl radicals such as the n-decyl radical, dodecyl radicals such as the n-dodecyl radical and octadecyl radicals such as the n-octadecyl radical, cycloalkyl radicals such as cyclopentyl, cyclohexyl, cycloheptyl and methylcyclohexyl radicals. The radical R is preferably a monovalent hydrocarbon radical having from 1 to 6 carbon atoms, e.g. the methyl, ethyl, n-propyl, isopropyl, n-butyl, sec-butyl, amyl and hexyl radicals, with particular preference being given to the methyl radical.

Preferred alkoxy radicals R are ones having from 1 to 6 carbon atoms, e.g. the methoxy, ethoxy, propoxy and n-butoxy radicals which may be additionally substituted by oxyalkylene radicals such as oxyethylene or oxymethylene radicals. Particular preference is given to the methoxy and ethoxy radicals. The alkyl radicals and alkoxy radicals R mentioned may also be substituted, for example by halogen, mercapto groups, epoxy-functional groups, carboxy groups, keto groups, enamine groups, amino groups, aminoethylamino groups, isocyanato groups, aryloxy groups, alkoxysilyl groups and hydroxy groups.

Suitable polymerizable groups R¹ are alkenyl radicals having from 2 to 8 carbon atoms. Examples of such polymerizable groups are the vinyl, allyl, butenyl and also acryloxyalkyl and methacryloxyalkyl groups, where the alkyl radicals contain from 1 to 4 carbon atoms. Preference is given to the vinyl group and the 3-methacryloxypropyl, acryloxymethyl and 3-acryloxypropyl groups.

Preference is given to α,ω-divinylpolydimethylsiloxanes, α,ω-di(3-acryloxypropyl)polydimethylsiloxanes, α,ω-di(3-methacryloxypropyl)polydimethylsiloxanes. Among silicones substituted by only one unsaturated group, preference is given to α-monovinylpolydimethylsiloxanes, α-mono(3-acryloxypropyl)-polydimethylsiloxanes, α-mono(acryloxymethyl)polydimethylsiloxanes, α-mono(3-methacryloxypropyl)polydimethylsiloxanes. In the monofunctional polydimethylsiloxanes, an alkyl or alkoxy radical, for example a methyl or butyl radical, is present at the other end of the chain.

Preference is also given to mixtures of linear or branched divinylpolydimethylsiloxanes with linear or branched monovinylpolydimethylsiloxanes and/or unfunctionalized polydimethylsiloxanes (the latter have no polymerizable group). The vinyl groups are located at the end of the chain. Examples of such mixtures are silicones of the solvent-free Dehesive®-6 series (branched) or Dehesive®-9 series (unbranched) from Wacker-Chemie GmbH. In the binary or ternary mixtures, the proportion of unfunctionalized polydialkyl-siloxanes is up to a maximum of 15% by weight, preferably up to 5% by weight; the proportion of monofunctional polydialkyl-siloxanes is up to 50% by weight; and the proportion of bifunctional polydialkylsiloxanes is at least 50% by weight, preferably at least 60% by weight, in each case based on the total weight of the silicone macromer.

The polymerizable silicone macromers described in EP-A 614924 are also suitable.

Most preferred silicone macromers are α,ω-divinylpolydimethylsiloxanes, 1-mono(3-methacryloxypropyl)polydimethylsiloxanes and α,ω-di(3-methacryloxypropyl)polydimethylsiloxanes.

The preparation of the prepolymer in step A) is carried out by means of free-radical polymerization as a bulk, solution, suspension or emulsion polymerization in an aqueous medium. The solution polymerization process and the suspension polymerization process are preferably used. Suitable solvents are esters such as methyl acetate and ethyl acetate, ketones such as acetone and methyl ethyl ketone, alcohols such as methanol, ethanol, t-butanol, isopropanol, or mixtures thereof.

The polymerization is usually carried out in a temperature range from 20° C. to 100° C., in particular from 40° C. to 80° C. The silicone macromer having ethylenically unsaturated groups is used in an amount of from 1 to 99% by weight, preferably from 5 to 60% by weight, in each case based on the total weight of silicone macromer and ethylenically unsaturated monomer. Initiation is effected by means of free-radical formers (initiators), which are preferably used in amounts of from 0.01 to 5.0% by weight, based on the total weight of silicone macromer and monomer. It is possible to employ a water-soluble or oil-soluble initiator or mixtures thereof.

Suitable water-soluble initiators are ones whose solubility in water under normal conditions is ≧10% by weight. Examples are water-soluble, inorganic peroxides such as ammonium, sodium, potassium peroxodisulphate or hydrogen peroxide, either alone or in combination with reducing agents such as sodium sulphite, sodium hydrogensulphite, sodium formaldehyde-sulphoxylate or ascorbic acid. It is also possible to use water-soluble organic peroxides, for example t-butyl hydroperoxide (TBHP), cumene hydroperoxide, usually in combination with reducing agents, or else water-soluble azo compounds.

Oil-soluble initiators are initiators whose solubility in water under normal conditions is ≦1% by weight. Representatives of the group of oil-soluble initiators which are used are initiators such as t-butyl peroxy-2-ethylhexanoate (TBPEH), t-butyl peroxypivalate (PPV), t-butyl peroxyneodecanoate (TBPND), dibenzoyl peroxide, t-amyl peroxypivalate (TAPPI), di(2-ethylhexyl) peroxydicarbonate (EHPC), 1,1-bis(t-butylperoxy)-3,3,5-trimethylcyclohexane and di(4-t-butylcyclohexyl) peroxydicarbonate. Further suitable oil-soluble initiators are azo initiators such as azobisisobutyronitrile (AIBN).

The copolymerization using gaseous monomers such as ethylene and vinyl chloride is carried out under superatmospheric pressure, generally in the range from 1 to 100 bar_{abs}.

If appropriate, the molecular weight can be regulated using the customary regulators, for example alcohols such as isopropanol, aldehydes such as acetaldehyde, chlorine-containing compounds, mercaptans such as n-dodecyl mercaptan, t-dodecyl mercaptan, mercaptopropionic acid (esters). To set the pH, it is possible to use pH-regulating compounds such as sodium acetate or formic acid in the preparation of the dispersion.

If the polymerization is carried out in the aqueous phase, the emulsifiers and protective colloids mentioned in the description of step C) can also be used for the purposes of stabilization.

Regardless of the polymerization process, the polymerization can be carried out with all constituents or individual constituents of the reaction mixture being initially charged, or with part of the constituents being initially charged and further amounts of the constituents or individual constituents of the reaction mixture being metered in, or by the feed stream process without an initial charge. After the polymerization is complete, the prepolymer is isolated. Depending on the polymerization process, this can be carried out in a known manner by means of filtration, precipitation or removal of the solvent by distillation.

The prepolymers obtained in this way are dissolved in one or more ethylenically unsaturated monomers in the next step. The proportion of prepolymer in the solution is preferably from 5 to 60% by weight, based on the total weight of prepolymer and ethylenically unsaturated monomer. Suitable ethylenically unsaturated monomers are the monomers from the group consisting of vinyl esters of unbranched or branched alkylcarboxylic acids having from 1 to 15 carbon atoms, methacrylic esters and acrylic esters of alcohols having from 1 to 15 carbon atoms, vinylaromatics, olefins, dienes and vinyl halides which have been mentioned above under step A) and also, if appropriate, additionally the above-mentioned auxiliary monomers in the amounts mentioned.

Preference is given to vinyl acetate; mixtures of vinyl acetate and ethylene; mixtures of vinyl acetate with further vinyl esters such as vinyl laurate, vinyl pivalate, vinyl 2-ethylhexanoate, vinyl esters of an alpha-branched carboxylic acid, in particular vinyl esters of Versatic acid (VeoVa9^{R}, VeoVa10^{R}), and, if appropriate, ethylene; mixtures of vinyl esters, ethylene and vinyl chloride, with preference being given to vinyl acetate and/or vinyl propionate and/or one or more copolymerizable vinyl esters such as vinyl laurate, vinyl pivalate, vinyl 2-ethylhexanoate, vinyl esters of an alpha-branched carboxylic acid, in particular vinyl esters of Versatic acid (VeoVa9^{R}, VeoVa10^{R}), being present as vinyl esters; vinyl ester/acrylic ester mixtures with vinyl acetate and/or vinyl laurate and/or vinyl esters of Versatic acid and acrylic esters, in particular butyl acrylate or 2-ethylhexyl acrylate, which may additionally contain ethylene.

Preference is also given to (meth)acrylic esters such as methyl methacrylate, n-butyl acrylate and/or 2-ethylhexyl acrylate and also mixtures thereof; and also styrene; mixtures of styrene with (meth)acrylic esters such as butyl acrylate, methyl methacrylate and/or 2-ethylhexyl acrylate; mixtures of 1,3-butadiene with methyl methacrylate and/or styrene. The mixtures mentioned may additionally contain the abovementioned auxiliary monomers in the amounts mentioned.

The solution of prepolymer and ethylenically unsaturated monomer is emulsified in water and preferably polymerized by a free-radical mechanism using the emulsion polymerization process. The polymerization is usually carried out in a temperature range from 20° C. to 100° C. Initiation is effected by means of free-radical formers (initiators), which are preferably used in amounts of from 0.01 to 5.0% by weight, based on the total weight of silicone organocopolymer and monomer. It is possible to employ a water-soluble or oil-soluble initiator or a mixture of such initiators. Suitable water-soluble and oil-soluble initiators are those which have been mentioned above.

To stabilize the aqueous dispersion, it is possible to use anionic and nonionic emulsifiers and also protective colloids, with these also being able to contain polymerizable groups. Preference is given to using nonionic or anionic emulsifiers, particularly preferably a mixture of nonionic and anionic emulsifiers. As nonionic emulsifiers, preference is given to using condensation products of ethylene oxide or propylene oxide with linear or branched alcohols having from 8 to 18 carbon atoms, alkylphenols or linear or branched carboxylic acids having from 8 to 18 carbon atoms, and also block copolymers of ethylene oxide and propylene oxide. Suitable anionic emulsifiers are, for example, alkylsulphates, alkylsulphonates, alkylarylsulphates and also sulphates or phosphates of condensation products of ethylene oxide with linear or branched alkyl alcohols, with such condensation products having from 3 to 60 EO units, alkylphenols and monoesters or diesters of sulphosuccinic acid. The amount of emulsifier is from 0.1 to 30% by weight, based on the total weight of monomer and prepolymer used.

If appropriate, protective colloids can also be used. Examples of suitable protective colloids are polyvinyl alcohols containing from 75 to 95 mol %, preferably from 84 to 92 mol %, of vinyl alcohol units; poly-N-vinyl amides such as polyvinyl-pyrrolidones; polysaccharides such as starches and celluloses and their carboxymethyl, methyl, hydroxyethyl, hydroxypropyl derivatives; synthetic polymers such as poly(meth)acrylic acid, poly(meth)acrylamide. It is also possible to use polyglycol ethers such as polyethylene glycol, polypropylene glycol or mixed polyalkylene oxides having ethylene oxide and propylene oxide groups. Particular preference is given to using the abovementioned polyvinyl alcohols. Preference is also given to the use of polyalkylene oxides having at least one polymerizable group and not more than two polymerizable groups such as the vinyl group or the allyl group. The protective colloids are generally used in an amount of from 0.1 to 30% by weight, based on the total weight of the monomer and prepolymer used.

If appropriate, the molecular weight can be controlled using the customary regulators, for example alcohols such as isopropanol, aldehydes such as acetaldehyde, chlorine-containing compounds, mercaptans such as n-dodecyl mercaptan, t-dodecyl mercaptan, mercaptopropionic acid (esters). To set the pH, it is possible to use pH-regulating compounds such as sodium acetate or formic acid in the preparation of the dispersion.

Regardless of the polymerization process, the polymerization can be carried out with or without use of seed lattices, with all constituents or individual constituents of the reaction mixture being initially charged, or with part of the constituents being initially charged and further amounts of the constituents or individual constituents of the reaction mixture being metered in, or by the feed stream process without an initial charge. The prepolymer is always introduced as a solution in the monomer.

The emulsifiers and protective colloids can be initially charged for the preparation of the dispersion, or can be metered in, or parts of them can be initially charged and the remainder be metered in. Here, the surface-active substances can be introduced alone or as a preemulsion with the comonomers containing the prepolymer dissolved therein.

In the copolymerization of gaseous monomers such as ethylene, the desired amount is introduced by setting a particular pressure. The pressure at which the gaseous monomer is introduced can be set to a particular value at the beginning and can decrease during the polymerization, or the pressure is kept constant during the entire polymerization. The latter embodiment is preferred.

After the polymerization is complete, an after-polymerization can be carried out by known methods to remove residual monomers, for example by means of an after-polymerization initiated by a redox catalyst. Volatile residual monomers and further volatile, nonaqueous constituents of the dispersion can also be removed by means of distillation, preferably under reduced pressure, and, if appropriate, with inert entrainer gases such as air, nitrogen or steam being passed through or over the dispersion.

The aqueous dispersions obtainable by the process of the invention have a solids content of from 20 to 70% by weight, preferably from 25 to 65% by weight. The solids content can also be set by subsequent addition of water after the emulsion polymerization has been concluded. To prepare water-redispersible polymer powders, the aqueous dispersions are, if appropriate after addition of protective colloids as atomization aids, dried, for example by means of fluidized-bed drying, freeze drying or spray drying. The dispersions are preferable spray dried. Spray drying is carried out in customary spray-drying units, with atomization being able to be effected by means of single-fluid, two-fluid or multi-fluid nozzles or by means of a rotating disc. The output temperature is generally in the range from 45° C. to 120° C., preferably from 60° C. to 90° C., depending on the unit, the T_{g} of the resin and the desired degree of drying. The atomization aid is generally used in a total amount of from 3 to 30% by weight, based on the polymeric constituents of the dispersion. Suitable atomization aids are the protective colloids mentioned above. A content of up to 1.5% by weight of antifoam, based on the base polymer, has frequently been found to be advantageous for atomization. To improve the blocking stability, the powder obtained can be provided with an antiblocking agent (anticaking agent), preferably in an amount of up to 30% by weight, based on the total weight of polymeric constituents. Examples of antiblocking agents are calcium carbonate or magnesium carbonate, talc, gypsum, silica, kaolins, silicates.

The procedure according to the invention makes it possible to obtain silicone-containing copolymers in which all of the silicone component is bound to the organic component, which is ensured by the separate preparation of the prepolymer. The dispersions obtained in this way have a series of advantages: advantageous particle size distribution, storage stability, no phase separation, no sweating-out of the silicone, no speck formation and excellent film formation. Films which do not smear and have a high cohesion and advantageous mechanical properties are obtained.

From the matrix with the organic polymer, the silicone in the form of the previously formed silicone organocopolymer can display its action, e.g. it can lead to release behaviour and hydrophobic behaviour. The action of the silicone organo-copolymer can also be brought about by thermal activation.

The copolymers in the form of their aqueous dispersions and water-redispersible powders are suitable for use in adhesives, coating compositions, also as protective coating for, for example, metals, films, wood, or as release coating or for paper treatment, e.g. for tissues, as binders for the consolidation of fibres or other particulate materials. They can also be used in the textile sector for textile treatment, coating, textile finishing and in the fabric care sector. They are also suitable as modifiers and as hydrophobicizing agents. They can also be used advantageously in the field of polishes. In addition, the dispersions can be used as release agents. They are also suitable as binders in the building sector for paints, adhesives and coating compositions, for example in tile adhesives and thermal insulation adhesives, and in particular for use in low-emission plastic emulsion paints and plastic emulsion renders, both for interior and exterior use. They can also be employed as additives, e.g. in surface coating compositions or in cosmetic formulations such as hair sprays, creams, lotions or shampoos.

Furthermore, the copolymers in the form of their aqueous dispersions and water-redispersible powders are suitable as binders for toners for producing silicone-modified toner particles.

The following examples serve to illustrate the invention without restricting it in any way.

### Preparation of the Prepolymer:

### EXAMPLE a

Prepolymer prepared by solution polymerization.

Composition: 33% by weight of polydimethylsiloxane and 67% by weight of vinyl acetate

51.05 kg of ethyl acetate, 8.01 kg of isopropanol, 983.4 g of an α,ω-divinyl-functionalized polydimethylsiloxane having 133 SiOMe₂ repeating units (silicone macromer Wacker VIPO 300®), 51.3 g of tert-butyl perpivalate (Initiator PPV) and 2.00 kg of vinyl acetate were placed in a 120 l stirred vessel provided with an anchor stirrer, reflux condenser and metering facilities. The stirred vessel was subsequently heated to 70° C. at a stirrer speed of 95 rpm. After the internal temperature of 70° C. had been reached, the introduction of initiator solution (4.03 kg of ethyl acetate and 199.3 g of PPV) at a rate of 819.0 g/h was commenced. Ten minutes after the commencement of the metered addition of initiator, the monomers (7.88 kg of silicone macromer Wacker VIPO 300® and 15.99 kg of vinyl acetate) were fed in at a rate of 5.97 kg/h. The metered addition of initiator extended over a period of 310 minutes, while the metered addition of the monomers ended 60 minutes earlier. After the end of both metered additions, the polymerization was continued at 70° C. for another 120 minutes. The 1-phase polymer solution obtained was subsequently distilled in the stirred vessel at 95° C. with addition of 1000 ml of water and was subsequently dried at 120° C. for 1 hour. After cooling to room temperature, a transparent resin was obtained.

Analyses: Composition of the silicone organocopolymer according to 1H-NMR spectroscopy: 33% by weight of silicone, 67% by weight of vinyl acetate; SC: 99.90%, GC analysis: residual VAc content <5 ppm; residual ethyl acetate 45 ppm; residual isopropanol 10 ppm, acid number 1.80 mgKOH/g, viscosity (Höppler, 10% strength solution in ethyl acetate)=1.16 mPas, SEC M_{w}=21 700 g/mol, Mₙ=4530 g/mol, polydispersity=4.79; T_{g}=24.3° C.

The 1H-NMR spectrum demonstrated that there were no longer any free double bonds present in the silicone organocopolymer. All of the silicone macromer has thus been copolymerized with the organic monomer and free silicone is no longer present.

### EXAMPLE b

Prepolymer prepared by suspension polymerization, composition: 30.0% by weight of polydimethyl siloxane (silicone), 70.0% by weight of styrene 237.35 kg of deionized water, 771.94 g of copper acetate (1% strength aqueous solution) and 10.29 kg of polyvinyl-pyrrolidone (5% strength aqueous solution) were placed in a 500 l stirred vessel provided with a stirrer, reflux condenser, metering facilities, heating facility (with temperature control) and nitrogen connection. The solution was stirred at 100 rpm. In the meantime, a mixture of 15.42 kg of an α-methacryloxypropyl-functionalized polydimethylsiloxane having 11 SiOMe₂ repeating units (Chisso FM 0711) and 15.42 kg of an α-methacryloxypropyl-functionalized polydimethylsiloxane having 63 SiOMe₂ repeating units (Chisso FM 0721) and 71.95 kg of styrene was prepared. A combination of two different initiators was added to this monomer mixture. Initiators used were 1.80 kg of t-butyl peroxyneodecanoate (95% by weight in aliphatics; half-life t1/2=1 h at 64° C.) and 1.75 kg of t-butyl perpivalate (75% by weight in aliphatics; half-life t1/2=1 h at 74° C.). The monomer mixture with the initiators was briefly stirred at room temperature and was then slowly added to the aqueous initial charge in the stirred vessel. The contents of the vessel were mixed by stirring at 100 rpm, resulting in the monomers being suspended in water. The temperature was subsequently increased to 55° C. and maintained for 4 hours. The temperature was then increased with a ramp of 0.1° C./min to 60° C. and maintained for 4 hours. The reaction mixture was then heated further with a temperature ramp of 0.1° C./min to 65° C. This temperature was maintained for 4 hours. The reaction mixture was then heated further with a temperature ramp of 0.1° C./min to 70° C. This temperature was maintained for 4 hours. The temperature was then increased with a ramp of 0.1° C./min to 75° C. and maintained for 4 hours. To complete the polymerization, the temperature was increased to 80° C. and maintained for 2 hours. The reaction mixture was subjected to a treatment with steam in order to drive out volatile compounds and was finally cooled to room temperature. The beads obtained were separated from the suspension medium, i.e. from the water, by means of a filtration step. The beads were washed a number of times with water and then dried. Highly transparent, hard beads were obtained.

Analyses: Composition according to 1H-NMR spectroscopy: 30.0% by weight of silicone, 70.0% by weight of styrene. Molecular weight M_{w} (weight average from GPC; eluent THF): 312 000 g/mol (based on polystyrene standards); polydispersity D: 6.5; glass transition temperature T_{g} (from DSC): 68° C.

The 1H-NMR spectrum demonstrated that there were no longer any free double bonds present in the silicone organocopolymer. All of the silicone macromer has thus been copolymerized with the organic monomer and free silicone is no longer present.

### Preparation of the Polymer Dispersions:

### Raw Materials Used:

Mersolat K30: Na alkylsulphonate having 12-14 carbon atoms in the alkyl radical.

Genapol X050: C₁₃ oxo alcohol ethoxylate with 5 EO

Texapon K12: Na dodecylsulphate

Genapol PF80: EO-PO block polymer with 80% of EO.

Brüggolith: sodium formaldehyde sulphoxylate (reducing agent)

Polyvinyl alcohol W25/140: polyvinyl alcohol having a viscosity of about 25 mPas (20° C., 4% strength solution, measured by the Höppler method) and a saponification number of 140 (mg of KOH/g of polymer) (degree of hydrolysis=88 mol %).

### EXAMPLE 1

622.0 g of W 25/140 (polyvinyl alcohol, 10% strength aqueous solution), 155.5 g of Genapol PF 80 (20% strength aqueous solution), 11.47 g of Mersolat K30 (30% strength aqueous solution) were placed in a 2 litre stirred apparatus provided with an anchor stirrer and metering facilities. A previously prepared solution of 311.0 g of a silicone organocopolymer having the composition 33.0% by weight of silicone, 67.0% by weight of vinyl acetate (prepared in Example a)), 2.62 g of Trigonox 23 (tert-butyl peroxyneodecanoate, 95% strength in aliphatics) in 311.0 g of vinyl acetate was added thereto. This initial charge was stirred at 300 rpm. The pH was adjusted to 5-5.5 by means of 10% strength formic acid.

The vessel was subsequently heated to 60° C.

The temperature of 60° C. was maintained for 2 hours while stirring at 300 rpm.

After the end of the polymerization, the dispersion was treated with steam (stripped) to minimize the residual monomer content and Hydrorol W was subsequently added as preservative. The dispersion was diluted with 185 g of water before being packed. A homogeneous and stable dispersion was obtained.

### Dispersion Analyses:

Solids content: 44.2%; pH: 4.1; Brookfield viscosity 20 (spindle 7): 28 200 mPas; MFT: 10° C.; glass transition temperature T_{g}: 19.2° C.; mean particle size: 1653.5 nm (Nanosizer); Coulter: Dₙ (number average particle size)=0.479 μm; Dᵥ (volume average particle size)=0.839 μm; surface area=8.5 m²/g.

### EXAMPLE 2

622.0 g of W 25/140 (polyvinyl alcohol, 10% strength aqueous solution), 146.0 g of Genapol PF 80 (20% strength aqueous solution), 10.77 g of Mersolat K30 (30% strength aqueous solution) were placed in a 2 litre stirred apparatus provided with an anchor stirrer. A previously prepared solution of 292.0 g of a silicone organocopolymer having the composition 33.0% by weight of silicone, 67.0% by weight of vinyl acetate (prepared in Example a)) in 292.0 g of vinyl acetate was added thereto. This initial charge was stirred at 300 rpm. The pH was adjusted to 5-5.5 by means of 10% strength formic acid. The vessel was subsequently heated to 60° C. and stirred at 300 rpm. As soon as the reactor was in thermal equilibrium, an 8.7% strength aqueous TBHP solution (tert-butyl hydroperoxide) was introduced at 12.4 g per hour and a 4.92% strength Brüggolith solution was introduced at 32.8 g per hour. The two metered additions proceeded for a period of 2 hours. After the end of the metered addition of TBHP and Brüggolith, the reaction mixture was maintained at 60° C. for 1 hour. After the end of the polymerization, the dispersion was treated with steam (stripped) to minimize the residual monomer content and Hydrorol W was subsequently added as preservative. A homogeneous and stable dispersion was obtained.

### Dispersion Analyses:

Solids content: 45.8%; pH: 3.9; Brookfield viscosity 20 (spindle 7): 83 000 mPas; MFT: 6° C.; glass transition temperature T_{g}: 20.2° C.; mean particle size: 557.7 nm (Nanosizer)

Coulter: Dₙ=0.268 μm; Dᵥ=0.810 μm; surface area 10.8 m²/g.

### EXAMPLE 3

116.74 g of W 25/140 (polyvinyl alcohol, 10% strength aqueous solution), 29.18 g of Genapol PF 80 (20% strength aqueous solution), 2.22 g of Mersolat K30 (30% strength aqueous solution) were placed in a 2 litre stirred apparatus provided with an anchor stirrer. A previously prepared solution of 23.35 g of a silicone organocopolymer having the composition 70.0% by weight of styrene and 30.0% by weight of silicone (prepared in Example b)) in 93.4 g of vinyl acetate was added thereto. This initial charge was stirred at 300 rpm. The pH was adjusted to 5-5.5 by means of 10% strength formic acid. The vessel was subsequently heated to 60° C. As soon as the reactor was in thermal equilibrium, an 8.7% strength TBHP solution (tert-butyl hydroperoxide) was introduced at 5.72 g per hour and a 4.94% strength Brüggolith solution was introduced at 15.15 g per hour. 20 minutes later, the introduction of a mixture (in the form of a preemulsion) of 150.0 g of water, 467.0 g of W 25/140 (polyvinyl alcohol, 10% strength aqueous solution), 116.74 g of Genapol PF 80 (20% strength aqueous solution), 8.87 g of Mersolat K30 (30% strength aqueous solution) and a previously prepared solution of 93.4 g of a silicone organocopolymer having the composition 70.0% by weight of styrene and 30.0% by weight of silicone (prepared in Example b)) in 373.6 g of vinyl acetate at a rate of 353.3 g per hour was commenced (metered addition of preemulsion). During the reaction, the mixture was stirred at 300 rpm. The total addition time for the metered addition of preemulsion was 3 hours. After the end of the metered addition of preemulsion, the introduction of TBHP and Brüggolith was continued for 1 hour. The dispersion was subsequently treated with steam (stripped) to minimize the residual monomer content and Hydrorol W was added as preservative. A homogeneous and stable dispersion was obtained.

### Dispersion Analyses:

Solids content: 43.4%; pH: 4.3; Brookfield viscosity 20 (spindle 7): 7900 mPas; glass transition temperatures T_{g}: T_{g}1=25.1° C., T_{g}2=66.3° C. (very low); mean particle size: 438.8 nm (Nanosizer); Coulter: Dₙ=0.339 μm; Dᵥ=0.521 μm; surface area=16.3 m²/g

### EXAMPLE 4

97.95 g of W 25/140 (polyvinyl alcohol, 10% strength aqueous solution), 24.49 g of Genapol PF 80 (20% strength aqueous solution), 1.86 g of Mersolat K30 (30% strength aqueous solution) were placed in a 2 litre stirred apparatus provided with an anchor stirrer. A previously prepared solution of 19.59 g of a silicone organocopolymer having the composition 70.0% by weight of styrene and 30.0% by weight of silicone (prepared in Example b)) in 78.36 g of methyl methacrylate was added thereto. The initial charge was stirred at 200 rpm. The pH was adjusted to 5-5.5 by means of 10% strength formic acid. The vessel was subsequently heated to 60° C. As soon as the reactor was in thermal equilibrium, an 8.7% strength TBHP solution (tert-butyl hydroperoxide) was introduced at 4.8 g per hour and a 4.92% strength Brüggolith solution was introduced at 12.7 g per hour. 20 minutes later, the introduction of a mixture (in the form of a preemulsion) of 225.29 g of water, 391.81 g of W 25/140 (polyvinyl alcohol, 10% strength aqueous solution), 97.95 g of Genapol PF 80 (20% strength aqueous solution), 7.44 g of Mersolat K30 (30% strength aqueous solution) and a previously prepared solution of 78.36 g of a silicone organocopolymer having the composition 70.0% by weight of styrene and 30.0% by weight of silicone (prepared in Example b)) in 313.45 g of methyl methacrylate at a rate of 370 g per hour was commenced (metered addition of preemulsion). During the reaction, the mixture was stirred at 200 rpm. The total addition time for the metered addition of preemulsion was 3 hours. After the end of the metered addition of preemulsion, the introduction of TBHP and Brüggolith was continued for 1 hour. The dispersion was subsequently treated with steam (stripped) to minimize the residual monomer content and Hydrorol W was added as preservative. The dispersion was diluted with 200 g of water before being packed. A homogeneous and stable dispersion was obtained.

### Dispersion Analyses:

Solids content: 33.0%; pH: 5.1; Brookfield viscosity 20 (spindle 5): 12 240 mPas; glass transition temperatures T_{g}: T_{g}1=68.3° C. (low), T_{g}2=104.1° C. (high); mean particle size: 637.2 nm (Nanosizer); Coulter: Dₙ=0.115 μm; Dᵥ=26.25 μm; surface area=9.06 m²/g.

### EXAMPLE 5

91.55 g of water, 91.55 g of W 25/140 (polyvinyl alcohol, 10% strength aqueous solution), 22.89 g of Genapol PF 80 (20% strength aqueous solution), 1.74 g of Mersolat K30 (30% strength aqueous solution) were placed in a 2 litre stirred apparatus provided with an anchor stirrer. A previously prepared solution of 18.31 g of a silicone organocopolymer having the composition 67.0% by weight of vinyl acetate and 33.0% by weight of silicone (prepared in Example a)) in 73.24 g of methyl methacrylate was added thereto. The pH was adjusted to 5-5.5 by means of 10% strength formic acid. The initial charge was stirred at 200 rpm. The vessel was subsequently heated to 60° C. As soon as the reactor was in thermal equilibrium, an 8.7% strength TBHP solution (tert-butyl hydroperoxide) was introduced at 4.5 g per hour and a 4.92% strength Brüggolith solution was introduced at 11.9 g per hour. 20 minutes later, the introduction of a mixture (in the form of a preemulsion) of 210.56 g of water, 366.19 g of W 25/140 (polyvinyl alcohol, 10% strength aqueous solution), 91.55 g of Genapol PF 80 (20% strength aqueous solution), 6.96 g of Mersolat K30 (30% strength aqueous solution) and a previously prepared solution of 73.24 g of a silicone organocopolymer having the composition 67.0% by weight of vinyl acetate and 33.0% by weight of silicone (prepared in Example a)) in 292.95 g of methyl methacrylate at a rate of 346.7 g per hour was commenced (metered addition of preemulsion). During the reaction, the mixture was stirred at 200 rpm. The total addition time for the metered addition of preemulsion was 3 hours. After the end of the metered addition of preemulsion, the introduction of TBHP and Brüggolith was continued for 1 hour. The dispersion was subsequently treated with steam (stripped) to minimize the residual monomer content and Hydrorol W was added as preservative. The dispersion was diluted with 200 g of water before being packed. A homogeneous and stable dispersion was obtained.

### Dispersion Analyses:

Solids content: 32.2%; pH: 4.9; Brookfield viscosity 20 (spindle 4): 5650 mPas; glass transition temperature T_{g}: 81.3° C. (broad); mean particle size: 1295.1 nm (Nanosizer); Coulter: Dₙ=0.104 μm; Dᵥ=32.41 μm; surface area=5.39 m²/g.

### EXAMPLE 6

91.55 g of water, 91.55 g of W 25/140 (polyvinyl alcohol, 10% strength aqueous solution), 22.89 g of Genapol PF 80 (20% strength aqueous solution), 1.74 g of Mersolat K30 (30% strength aqueous solution) were placed in a 2 litre stirred apparatus provided with an anchor stirrer. A previously prepared solution of 18.31 g of a silicone organocopolymer having the composition 70.0% by weight of styrene and 30% by weight of silicone (prepared in Example b)) in 36.62 g of methyl methacrylate and 36.62 g of butyl acrylate was added thereto. The initial charge was stirred at 200 rpm. The pH was adjusted to 5-5.5 by means of 10% strength formic acid. The vessel was subsequently heated to 60° C. As soon as the reactor was in thermal equilibrium, an 8.7% strength TBHP solution (tert-butyl hydroperoxide) was introduced at 4.5 g per hour and a 4.92% strength Brüggolith solution was introduced at 11.88 g per hour. 20 minutes later, the introduction of a mixture (in the form of a preemulsion) of 210.56 g of water, 366.19 g of W 25/140 (polyvinyl alcohol, 10% strength aqueous solution), 91.55 g of Genapol PF 80 (20% strength aqueous solution), 6.96 g of Mersolat K30 (30% strength aqueous solution) and a previously prepared solution of 73.24 g of a silicone organocopolymer having the composition 70.0% by weight of styrene and 30.0% by weight of silicone (prepared in Example b)) in 146.48 g of methyl methacrylate and 146.48 g of butyl acrylate at a rate of 346.7 g per hour was commenced (metered addition of preemulsion). During the reaction, the mixture was stirred at 200 rpm. The total addition time for the metered addition of preemulsion was 3 hours. After the end of the metered addition of preemulsion, the introduction of TBHP and Brüggolith was continued for 1 hour. The dispersion was subsequently treated with steam (stripped) to minimize the residual monomer content and Hydrorol W was added as preservative. A homogeneous and stable dispersion was obtained.

### Dispersion Analyses:

Solids content: 37.54%; pH: 4.9; Brookfield viscosity 20 (spindle 4): 12 300 mPas; glass transition temperature T_{g}: 10.4° C.; mean particle size: 841.4 nm (Nanosizer); Coulter: Dₙ=0.257 μm; Dᵥ=1.643 μm; surface area=11.8 m²/g.

### COMPARATIVE EXAMPLE 7

1.29 kg of water, 2.67 kg of W 25/140 (polyvinyl alcohol, 10% strength aqueous solution), 101.55 g of Genapol X 050 (100% strength), 115.66 g of Texapon K12 (10% strength aqueous solution), 4.19 g of sodium acetate, 641.34 g of vinyl acetate and 427.56 g of an α,ω-divinyl-functionalized polydimethyl-siloxane having 133 SiOMe₂ repeating units (silicone macromer Wacker VIPO 300®) were placed in a 20 litre pressure autoclave. The pH was adjusted to 5 by means of 10% formic acid. In addition, 10 ml of Trilon B (EDTA; 2% strength aqueous solution) and 31 ml of iron ammonium sulphate (1% strength solution) were added. The vessel was heated to 70° C. and pressurized with 8 bar of nitrogen. As soon as the reactor was in thermal equilibrium, a 5.8% strength ammonium persulphate solution (APS solution) was introduced at 84 g per hour and a 2.68% strength sodium sulphite solution was introduced at 176 g per hour. 25 minutes later, introduction of a mixture of 2.57 kg of vinyl acetate and 1.71 kg of VIPO 300 at a rate of 2140 g per hour was commenced (metered addition of monomers). At the same time, an emulsifier mixture was introduced at a metering rate of 625 g per hour. The emulsifier mixture comprised 385.32 g of water, 406.18 g of Genapol X 050, and 462.62 g of Texapon K12 (10% strength aqueous solution). The total addition time for the metered addition of monomers and the metered addition of emulsifiers was 2 hours. 20 minutes after the beginning of the reaction, the rate of addition of the APS solution was increased to 126 g per hour, and that of the Na sulphite solution was increased to 262 g per hour. After the end of the metered addition of monomers and the metered addition of emulsifiers, the introduction of APS and Na sulphite was continued for 1 hour. After depressurization, the dispersion was treated with steam (stripped) to minimize the residual monomer content and Hydrorol W was subsequently added as preservative.

### Dispersion Analyses:

Solids content: 50.5%; pH: 5.3; Brookfield viscosity 20 (spindle 4): 1040 mPas; MFT: 0° C.; glass transition temperature T_{g}: 18.4° C.; mean particle size: 452.5 nm (Nanosizer); Coulter: Dₙ=0.124 μm; Dᵥ=1.697 μm; surface area=14.7 m²/g. Soxhlet extraction: residue from completely evaporated eluate 1.94 g=38.8% (extracted from 5 g of a dried dispersion film with cyclohexane).

Comparative Example 7 shows that conventional emulsion polymerization, in this case of vinyl acetate with silicone macromer, results in insufficient bonding of the silicone macromers to the organic monomer, which is also described in the literature. Here, 38.8% of the constituents could be washed out of a dried dispersion film in the extraction. 1H-NMR spectroscopy demonstrated that the extractable constituents here were mostly free silicone macromer. The process of the invention eliminates this problem and all the disadvantages resulting therefrom. The process of the present invention ensures that the organic monomer is entirely bound to the silicone macromer, i.e. free silicone is no longer present. This is achieved by preparing the silicone organocopolymer separately beforehand by polymerization.

1-11. (canceled)

12. A process for preparing silicone-modified polymers of ethylenically unsaturated monomers in the form of an aqueous polymer dispersions or water-redispersible polymer powder, comprising
A) preparing a prepolymer by copolymerizing of one or more ethylenically unsaturated monomers selected from the group consisting of vinyl esters of optionally branched C₁₋₁₅ alkylcarboxylic acids, (meth)acrylic esters of C₁₋₁₅ alcohols, vinylaromatics, olefins, dienes and vinyl halides, with at least one silicone macromer bearing ethylenically unsaturated groups, to form a prepolymer and isolating the prepolymer, wherein the silicone macromer is selected from the group consisting of linear, branched, cyclic and three-dimensionally crosslinked polysiloxanes having at least 10 siloxane repeating units and at least one free-radically polymerizable functional group and mixtures thereof,
B) dissolving the prepolymer in one or more ethylenically unsaturated monomers,
C) emulsifying the solution formed in B) in water and polymerizing by a free-radical mechanism to form an aqueous dispersions of silicone-modified polymers, and
D) optionally, drying the aqueous dispersion of silicone-modified polymers.

13. The process of claim 12, wherein the preparation of the prepolymer in step A) is carried out by means of free-radical solution polymerization.

14. The process of claim 12, wherein the preparation of the prepolymer in step A) is carried out by means of free-radical suspension polymerization.

15. The process of claim 12, wherein a free-radical polymerization using the emulsion polymerization process is carried out in step C).

16. In an adhesive composition, coating composition, binder composition for the consolidation of fibres or other particulate materials, textile treatment composition, or hydrophobicizing composition, wherein a polymer dispersion or redispersible polymer powder is employed, the improvement comprising incorporating into said composition, a silicone-modified polymer prepared by the process of claim 12.

17. In a paint composition, adhesive composition, or coating composition wherein a polymer dispersion or redispersible polymer powder is employed, the improvement comprising incorporating into said composition at least one silicone-modified polymer dispersion or redispersible polymer powder prepared by the process of claim 12.

18. In a cosmetic composition wherein a polymer dispersion or redispersible polymer powder is employed, the improvement comprising incorporating into said composition at least one silicone-modified polymer dispersion or redispersible polymer powder prepared by the process of claim 12.

19. In a silicone modified toner composition wherein a binder is employed, the improvement comprising incorporating into said composition at least one binder which comprises a silicone-modified polymer dispersion or redispersible polymer powder prepared by the process of claim 12.
